(19) United States
(12) Patent Application Publication
(10) Pub. No.: US 2005/0053558 A1
(43) Pub. Date: Mar. 10, 2005

(54) Sleep inducing toothpaste made with natural herbs and a natural hormone

(76) Inventor: Arthur Zuckerman, New York, NY (US)

Correspondence Address: EZRA SUTTON, PLAZA 9, 900 ROUTE 9, WOODBRIDGE, NJ, 07095, US

(21) Appl. No.: 10/969,586

(22) Filed: Oct. 20, 2004

Related U.S. Application Data
(63) Continuation-in-part of application No. 10/391,004, filed on Mar. 18, 2003.

(51) Int. Cl. A61K 7/26; A61K 35/78

(52) U.S. Cl. 424/58; 424/733; 424/745

(57) ABSTRACT

A toothpaste composition for inducing sleep while simultaneously promoting intraoral cleanliness, which includes toothpaste base ingredients and at least one sleep-inducing natural herb or hormone. The sleep-inducing natural herbs and hormone are selected from the group consisting of lemon balm, passion flower, and valerian, and the hormone melatonin. The sleep-inducing natural herbs or hormone are in a range of 0.010% to 2.0% by weight of the composition.

### RELATED APPLICATION

This is a continuation-in-part of application Ser. No. 10/391,004 filed on Mar. 18, 2003.

### FIELD OF THE INVENTION

Natural herbs and a natural hormone melatonin in combination with toothpaste is used at night to clean teeth and improve sleep. The expected dose of toothpaste is calculated at 2 grams. The ingredients have been assessed for the range of daily doses for best effects, toxicity in normal range, recommended proportion of each, and water solubility of key constituents.

### BACKGROUND OF THE INVENTION

It is an object of the present invention to provide a sleep-inducing toothpaste, dental cream, mouth spray or mouth wash which includes sleep-inducing natural herbs and a natural hormone.

It is a further object of the present invention to provide a sleep-inducing toothpaste which includes toothpaste base ingredients and natural herbs being lemon balm, passion flower, valerian and the natural hormone melatonin.

This new combination of toothpaste and the natural hormone melatonin has never been used before for inducing sleep and reducing jet lag.

### SUMMARY OF THE INVENTION

A toothpaste composition is provided for inducing sleep while simultaneously promoting intraoral cleanliness, which includes toothpaste base ingredients and at least one sleep-inducing natural herb or hormone. The sleep-inducing natural herbs and hormone are selected from the group consisting of the natural herbs lemon balm, passion flower, and valerian, and the natural hormone melatonin. The sleep-inducing natural herbs and hormone are in a range of 0.010% to 2.0% by weight of the composition, which may be in the form of a toothpaste dental cream, mouth wash or mouth spray.

### DESCRIPTION OF THE PREFERRED EMBODIMENTS

The toothpaste composition has base ingredients which comprise a combination of known amounts of: vegetable glycerin; sorbitol; hydrated silica; purified water; xylitol; carrageenan; sodium lauryl sulfate; titanium dioxide; propylparaben; methylparaben; sodium benzoate; and a flavoring agent.

The sleep-inducing natural herbs include the following:

A) Name: Lemon Balm

Scientific Name: Melissa officinalis

Family: Lamiacease, the Mint Family

Parts used: Herb, leaves, volatile oil and polyphenols.

Lemon Balm comprises substantially 0.5% to 2.0% by weight of the toothpaste composition and preferably comprises substantially 1.0% by weight of the composition.

In the proposed toothpaste lemon balm has potential for the treatment of:

insomnia

headaches

restlessness

toothache

viral sores

minor digestive spasm

Daily dose range for prime efficacy:

2-3 grams of the ground, dried herb in capsules or tablets, 1-3 times a day.

1:5 tincture: 2-6 ml, 1-3 times per day, best between or before means or one daily dose at bedtime.

1:2 tincture: 1-3 ml, as needed, or one daily dose at bedtime.

Tea: 1 ounce of leaves, stems, flowers to a pint of water. Boil water, pour over herb, steep 5 to 10 minutes, drink 1-2 cups or more per day.

Recommended Percent in Toothpaste Formula: 1% due to Lemon Balm's potent flavor and its mild sedative properties. When bruised, the aerial parts smell like lemon and mint. Fresh plant material may be used if it meets pharmacopoeia standards when-a-sample is dried.

Safety and Toxicity in Recommended Dosage Range:

No restrictions for ingestion or topical use are found.

US: Generally recognized as safe

UK: General sales list

Canada: approved as an over-the-counter drug

France: Traditional Medicine

Germany: Commission E approved as an over the counter drug.

Active Compounds:

Essential oil 0.06-0.375% with monoterpene aldehydes geranial & neral (Citral a & b), citronellal

Flavonoids apigenin, luteolin, kaempferol, quercetin

Glycosides

Up to 4% rosmarinic acid, anti-inflammatory and antioxidant

Ursolic, oleanolic acids

Sedative effects long appreciated in folk medicine have been confirmed only in animal experiments using water/alcohol extracts given by injection.

B) Name: Passion Flower

Scientific Name: Passiflora incarnata

Family: Passifloraceae

Parts Used: The leaves, stems, and flower before the vine fruits.

In the proposed toothpaste Passionflower has potential for the treatment of:

Insomnia

Muscle spasm

Pain

Emotional upset

Viral sores

Passion Flower comprises substantially 0.5% to 2.0% by weight of the toothpaste composition and preferably comprises substantially 1.0% by weight of the composition. Passion flower has a depressant effect on the Central Nervous System. The herb is used for its sedative properties, to lower blood pressure, and prevent tachycarduia. The alkaloid and flavonoids have both been reported to have sedative activity in animals.

Many of the flavonoids, such as apigenin, are well-known for pharmacological activity, particularly anti-spasmodic and anti-inflammatory activities. It is the herb of choice for treating intransigent insomnia.

Daily dose range for prime efficacy:

2-4 grams of the ground, dried herb in capsules or tablets, 1-3 times a day.

1:5 tincture: 6-12 ml, 1-3 times per day, or one daily dose all at bedtime.

1:2 tincture: 2-4 ml, as needed, or one daily dose all at bedtime.

Recommended Percent in Toothpaste Formula: 1.0%

Safety and Toxicity in Recommended Dosage Range:

Passion flower is generally safe and has not been found to negatively interact with other sedative drugs. The American Botanical Safety Handbook lists Passion Flower as category 1—no restrictions.

Active Compounds:

Alkaloids: harmine, harman, harmol, harmaline, harmalol, and passaflorine.

No less than 8% flavonoids: apigenin and various glycosides, homo-orientin, isovitexin, kaempferol, luteolin, orientin, quercitin, rutin, saponaretin, saponarin and vitexin. The flavonoids in passion flower are the primary constituents responsible for its relaxing and anti-anxiety effects.

C) Name: Valerian

Scientific Name: Valeriana officinalis

Family: Valerianaceae

Parts Used: Root and rhizome.

In the proposed toothpaste Valerian has potential for the treatment of:

Insomnia

Pain

Blood Pressure

Valerian comprises substantially 0.25% to 1.0% by weight of the toothpaste composition and preferably comprises substantially 0.5% by weight of the composition.

Daily dose range for prime efficacy:

A wide range experimentally and in clinical trials exists, from a minimum of 60-120 mg per dose up to 300-500 mg of the ground, dried herb in capsules or tablets, 1-3 times a day or one daily dose 1 hour before bedtime.

Recommended Percent in Toothpaste Formula: 0.5% This amount reflects this herb's primacy in an oral, water-extracted, herbal formula designed for helping sleep.

Safety and Toxicity in Recommended Dosage Range:

No adverse effects are expected when taken within recommended dosage ranges. The FDA lists valerian as generally safe.

Valerian has a long history of exceptional safety, which has been confirmed by clinical studies.

Active Compounds:

0.35-1% essential oils (not less than 5 ml/kg) including monoterpenes (borneol), sesquiterpenes (beta-bisabolene, valerenal in fresh root), esters of valerianic, isovalerianic acid Iridoids, 0.5%-2%, a.k.a. valepotriates and decomposition products including valtrate, isovlatrate, dihidrovaltrate, acevaltrate

Valerenic acid, its derivatives

Aminos, lignans

The essential oil (aroma and flavor) contribute perhaps ⅓ to the sedating properties of the herb. Many constituents are thought to synergize for its sedative effects. Central nervous system sedation is regulated by receptors in the brain known as GABA-A receptors. Valerian weakly binds to these receptors for a sedating effect.

Clinical studies have shown that people taking valerian had shown significantly improved sleep quality without morning grogginess. Some researchers have compared valerian to benzodiazepines such as Valium (to which it bears no relation, either etymologically or as a source for drug development). However, valerian is a much milder and safer sedative. Unlike Valium, valerian is not addictive or does not promote dependency. Valerian's sedative effect is not significantly exaggerated by alcohol and barbiturates, unlike Valium. Valerian extract at a dose of 50 to 100 mg taken two or three times daily has been shown to relieve performance anxiety and the stress of driving in heavy traffic. Larger doses of valerian extract may be necessary for patients who have been using benzodiazepine prescriptions for anxiety.

D) Name: Melatonin

The chemical symbol for the natural hormone Melatonin is:

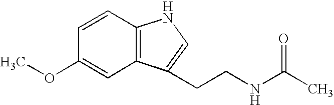

In the proposed toothpaste has potential for the treatment of:

Sleep disorders and jet lag.

Daily dose range for prime efficacy:

½ to 10 mg, thirty minutes before retiring. For sleeplessness due to low melatonin, the dosage range is 12 to 10 mg. The lower dose may be taken for at least three days, then gradually increased by 1 to 2 mg every three nights up to 10 mg if results are not what expected. Higher doses, 20 mg up to 50 mg, have been used for patients on chemotherapy.

Recommended Percent in Toothpaste Formula:

Melatonin comprises substantially 0.010% to 1.0% by weight of the toothpaste composition and preferably comprises substantially 0.20% by weight of the composition.

Percent depends on milligrams one dose of toothpaste can provide. The more there is the better the change that some will be absorbed orally. In a 2 gram dose of toothpaste 10 mg of melatonin may be the upper limit that can be accommodated. Though this may be effective as much as possible, up to 10 mg, is recommended since toothpaste isn't swallowed.

Safety and Toxicity in Recommended Dosage Range:

No toxicity is expected though studies of melatonin's safety are limited, with isolated reports of exacerbation of depression and fatigue.

History:

Melatonin has been called the body's own natural sleeping pill. It plays a key role in the sleep cycle by helping one fall asleep. Low melatonin levels can cause sleep-onset insomnia. The body changes serotonin into melatonin, a hormone stored in the pineal gland. The pineal releases melatonin only when the level of light is low. For most people melatonin is secreted only at night, during sleep unless lights are left on. The presence of light is a sign to the brain to shut down melatonin production.

### PREFERRED COMPOSITIONS OF THE INVENTION

A toothpaste composition for inducing sleep while simultaneously promoting intraoral cleanliness includes the following ingredients:
a) toothpaste base ingredients;
b) at least one sleep-inducing herb being selected from the group consisting of lemon balm, passion flower and valerian; and the hormone melatonin; and
c) wherein the lemon balm comprises an effective dosage in the range of 0.50% to 2.0% by weight of the toothpaste composition; the passion flower comprises an effective dosage in the range of 0.50% to 2.0% by weight of the toothpaste composition; the valerian comprises an effective dosage in the range of 0.25% to 1.0% by weight of the toothpaste composition; and the hormone melatonin comprises an effective dosage in the range of 0.010% to 1.0% by weight of the toothpaste composition.

A toothpaste composition for inducing sleep while simultaneously promoting intraoral cleanliness includes the following ingredients:
a) toothpaste base ingredients;
b) at least one sleep-inducing herb being selected from the group consisting of lemon balm, passion flower and valerian; and the hormone melatonin; and
c) wherein the lemon balm comprises an effective dosage of 1.0% by weight of the toothpaste composition; the passion flower comprises an effective dosage of 1.0% by weight of the toothpaste composition; the valerian comprises an effective dosage of 0.50% by weight of the toothpaste composition; and the hormone melatonin comprises an effective dosage of 0.015% by weight of the toothpaste composition.

### ADVANTAGES OF THE PRESENT INVENTION

Accordingly, an advantage of the present invention is that it provides for a sleep-inducing toothpaste made with natural herbs and a natural hormone melatonin being used at night to clean teeth and improve sleep by the user.

Another advantage of the present invention is that it provides for a sleep-inducing dental cream, mouth wash or mouth spray which includes sleep-inducing natural herbs and a natural hormone.

Another advantage of the present invention is that it provides for a sleep-inducing toothpaste which includes toothpaste base ingredients, natural herbs being lemon balm, passion flower, valerian and the natural hormone melatonin.

Another advantage of the present invention is that it provides for a sleep-inducing toothpaste which includes toothpaste base ingredients, natural herbs being lemon balm and valerian and the natural hormone melatonin.

Another advantage of the present invention is that it provides for a sleep-inducing toothpaste which includes toothpaste base ingredients, natural herbs being passion flower and valerian and the natural hormone melatonin.

Another advantage of the present invention is that it provides for a sleep-inducing toothpaste which includes toothpaste base ingredients, the natural herb valerian and the natural hormone melatonin.

A further advantage of the present invention is that it provides for a sleep-inducing toothpaste that is effective in producing a restful sleep to the user, wherein the natural herbs and the natural hormone melatonin are present in a range of 0.01% to 1.0% by weight of the toothpaste composition.

A further advantage of the present invention is that it provides for a sleep-inducing toothpaste that is effective in reducing jet lag after a long airplane trip.

A still further advantage of the present advantage is that it provides for a sleep inducing toothpaste that can be mass produced in an automated and economical manner and is readily affordable by the consumer.

A latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

1. A toothpaste composition for inducing sleep while simultaneously promoting intraoral cleanliness, comprising;
a) toothpaste base ingredients; and
b) at least one sleep-inducing herb being selected from the group consisting of lemon balm, passion flower and valerian; and the hormone melatonin.

2. A toothpaste composition in accordance with claim 1, wherein said toothpaste base ingredients comprise a combination of effective amounts of:
glycerin; sorbitol; hydrated silica; purified water; xylitol; carrageenan; sodium lauryl sulfate; titanium dioxide; propylparaben; methylparaben; sodium benzoate; and a flavoring agent.

3. A toothpaste composition in accordance with claim 1, wherein said sleep inducing natural herbs and said hormone melatonin are present in a range of 0.01% to 1.0% by weight of said toothpaste composition.

4. A toothpaste composition in accordance with claim 1, wherein said toothpaste composition is in the form of a dental cream, a mouth wash or a mouth spray.

5. A toothpaste composition for inducing sleep while simultaneously promoting intraoral cleanliness, comprising;
a) toothpaste base ingredients;
b) at least one sleep-inducing herb being selected from the group consisting of lemon balm, passion flower and valerian; and the hormone melatonin; and
c) wherein said lemon balm comprises an effective dosage in the range of 0.50% to 2.0% by weight of said toothpaste composition; said passion flower comprises an effective dosage in the range of 0.50% to 2.0% by weight of said toothpaste composition; said valerian comprises an effective dosage in the range of 0.25% to 1.0% by weight of said toothpaste composition; and said hormone melatonin comprises an effective dosage in the range of 0.010% to 1.0% by weight of said toothpaste composition.

6. A toothpaste composition in accordance with claim 5, wherein said lemon balm comprises an effective dosage of 1.0% by weight of said toothpaste composition; said passion flower comprises an effective dosage of 1.0% by weight of said toothpaste composition; said valerian comprises an effective dosage of 0.50% by weight of said toothpaste composition; and said hormone melatonin comprises an effective dosage of 0.015% by weight of said toothpaste composition.

7. A toothpaste composition for inducing sleep while simultaneously promoting intraoral cleanliness, comprising;
a) toothpaste base ingredients;
b) at least one sleep-inducing herb being selected from the group consisting of passion flower and valerian; and the hormone melatonin; and
c) wherein said passion flower comprises an effective dosage in the range of 0.50% to 2.0% by weight of said toothpaste composition; said valerian comprises an effective dosage in the range of 0.25% to 1.0% by weight of said toothpaste composition; and said hormone melatonin comprises an effective dosage in the range of 0.010% to 1.0% by weight of said toothpaste composition.

8. A toothpaste composition in accordance with claim 7, wherein said passion flower comprises an effective dosage of 1.0% by weight of said toothpaste composition; said valerian comprises an effective dosage of 0.50% by weight of said toothpaste composition; and said hormone melatonin comprises an effective dosage of 0.20% by weight of said toothpaste composition.

9. A toothpaste composition for inducing sleep while simultaneously promoting intraoral cleanliness, comprising;
a) toothpaste base ingredients;
b) at least one sleep-inducing herb being selected from the group consisting of lemon balm and valerian; and the hormone melatonin; and
c) wherein said passion flower comprises an effective dosage in the range of 0.50% to 2.0% by weight of said toothpaste composition; said valerian comprises an effective dosage in the range of 0.25% to 1.0% by weight of said toothpaste composition; and said hormone melatonin comprises an effective dosage in the range of 0.010% to 1.0% by weight of said toothpaste composition.

10. A toothpaste composition in accordance with claim 9, wherein said passion flower comprises an effective dosage of 1.0% by weight of said toothpaste composition; said valerian comprises an effective dosage of 0.50% by weight of said toothpaste composition; and said hormone melatonin comprises an effective dosage of 0.20% by weight of said toothpaste composition.

11. A toothpaste composition for inducing sleep while simultaneously promoting intraoral cleanliness, comprising;
a) toothpaste base ingredients;
b) a sleep-inducing herb being valerian; and a hormone melatonin; and
c) wherein said valerian comprises an effective dosage in the range of 0.25% to 1.0% by weight of said toothpaste composition; and said hormone melatonin comprises an effective dosage in the range of 0.010% to 1.0% by weight of said toothpaste composition.

12. A toothpaste composition in accordance with claim 11, wherein said valerian comprises an effective dosage of 0.50% by weight of said toothpaste composition; and said hormone melatonin comprises an effective dosage of 0.20% by weight of said toothpaste composition.

13. A unique method of inducing sleep and reducing jet lag using a new combination of a toothpaste composition and the hormone melatonin, comprising the steps of:
a) administering to a human a conventional dosage of a toothpaste composition having toothpaste base ingredients, and including an effective dosage of a hormone melatonin being in the range of 0.010% to 1.0% by weight of said toothpaste composition.

14. A unique method of inducing sleep and reducing jet lag in accordance with claim 13, wherein the step of administering includes an effective dosage of said hormone melatonin in the range of 0.15% to 1.0% by weight of said toothpaste composition.

15. A method of inducing sleep using a toothpaste composition, comprising the steps of:
a) administering to a human a conventional dosage of a toothpaste composition having toothpaste base ingredients, and including an effective dosage of at least one sleep-inducing herb being selected from the group consisting of lemon balm, passion flower and valerian; and
b) administering said effective dosage of said lemon balm being in the range of 0.50% to 2.0% by weight of said toothpaste composition; said passion flower being in the range of 0.50% to 2.0% by weight of said toothpaste composition; or said valerian being in the range of 0.25% to 1.0% by weight of said toothpaste composition.
